(19) United States
(12) Patent Application Publication
(10) Pub. No.: US 2008/0207577 A1
(43) Pub. Date: Aug. 28, 2008

(54) Combination I

(75) Inventors: Nigel Boughton-Smith, Loughborough (GB); Martin Braddock, Loughborough (GB)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 11/995,264

(22) PCT Filed: Jul. 10, 2006
(86) PCT No.: PCT/SE06/00863

§ 371 Date: Jan. 10, 2008

(30) Foreign Application Priority Data
Jul. 11, 2005 (SE) 0501646-4

(51) Int. Cl. A61K 31/58; A61K 31/56; A61P 11/00; A61P 11/06

(52) U.S. Cl. 514/174; 514/178

(57) ABSTRACT

The invention provides a pharmaceutical product, kit or composition comprising a first active ingredient which is a P2X₇ receptor antagonist, and a second active ingredient which is a corticosteroid, of use in the treatment of respiratory diseases such as chronic obstructive pulmonary disease and asthma.

The present invention relates to combinations of pharmaceutically active substances for use in the treatment of respiratory diseases, especially chronic obstructive pulmonary disease (COPD) and asthma.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include corticosteroids. Corticosteroids (also known as glucocorticosteroids or glucocorticoids) are potent anti-inflammatory agents. Whilst their exact mechanism of action is not clear, the end result of corticosteroid treatment is a decrease in the number, activity and movement of inflammatory cells into the bronchial submucosa, leading to decreased airway responsiveness. Corticosteroids may also cause reduced shedding of bronchial epithelial lining, vascular permeability, and mucus secretion.

Whilst corticosteroid treatment can yield important benefits, the efficacy of these agents is often far from satisfactory, particularly in COPD. Moreover, whilst the use of steroids may lead to therapeutic effects, it is desirable to be able to use steroids in low doses to minimise the occurrence and severity of undesirable side effects that may be associated with regular administration. Recent studies have also highlighted the problem of the acquisition of steroid resistance amongst patients suffering from respiratory diseases. For example, cigarette smokers with asthma have been found to be insensitive to short term inhaled corticosteroid therapy, but the disparity of the response between smokers and non-smokers appears to be reduced with high dose inhaled corticosteroid (Tomlinson et al., Thorax 2005; 60:282-287). Hence there is a pressing medical need for new therapies against respiratory diseases such as COPD and asthma, in particular for therapies with disease modifying potential.

Combination products comprising a corticosteroid and a β₂ adrenoceptor agonist are available. One such product is a combination of budesonide and formoterol fumarate (marketed by AstraZeneca under the tradename Symbicort®), which has proven to be effective in controlling asthma and COPD, and improving quality of life in many patients.

In view of the complexity of respiratory diseases such as asthma and COPD, it is unlikely that any one mediator can satisfactorily treat the disease alone. Moreover, whilst combination treatments using a corticosteroid and a β₂ adrenoceptor agonist deliver significant patient benefits, there remains a medical need for new therapies against respiratory diseases such as asthma and COPD, in particular for therapies with disease modifying potential.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

The present invention provides a pharmaceutical product comprising, in combination, a first active ingredient which is a P2X₇ receptor antagonist, and a second active ingredient which is a corticosteroid.

A beneficial therapeutic effect may be observed in the treatment of respiratory diseases if a P2X₇ receptor antagonist is used in combination with a corticosteroid. The beneficial effect may be observed when the two active substances are administered simultaneously (either in a single pharmaceutical preparation or via separate preparations), or sequentially or separately via separate pharmaceutical preparations.

The pharmaceutical product of the present invention may, for example, be a pharmaceutical composition comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

The pharmaceutical product of the present invention comprises, as a first active ingredient, a P2X₇ receptor antagonist. An antagonist of the P2X₇ receptor is a compound or other substance that is capable of preventing, whether fully or partially, activation of the P2X₇ receptor.

Methods for assaying for P2X₇ receptor antagonism are known in the art, for example from WO 01/42194 which describes an assay based on the observation that when the P2X₇ receptor is activated using a receptor agonist in the presence of ethidium bromide (a fluorescent DNA probe), an increase in the fluorescence of intracellular DNA-bound ethidium bromide is observed. Thus, an increase in fluorescence can be used as a measure of P2X₇ receptor activation and therefore to quantify the effect of a compound or substance on the P2X₇ receptor.

In WO 01/42194, the assay is carried out by taking a 96-well flat bottomed microtitre plate and filling the wells with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M benzoylbenzoyl adenosine triphosphate (bbATP, a known P2X₇ receptor agonist), and 25 μl of the high potassium buffer solution containing 3×10⁻⁵M test compound. The plate is covered with a plastics sheet and incubated at 37° C. for one hour. The plate is then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) are used separately in the test as controls. From the readings obtained, a pIC₅₀ figure is calculated for the test compound, this figure being the negative logarithm of the concentration of test compound necessary to reduce the bbATP agonist activity by 50%. A pIC₅₀ figure greater than 5.5 is normally indicative of an antagonist.

Examples of P2X₇ receptor antagonists which may be used in accordance with the present invention include adamantyl derivatives having P2X₇ receptor antagonist properties, as for example described in WO 00/61569, WO 01/42194, WO 01/44170 and WO 03/41707, the entire contents of which are incorporated herein by reference.

In an embodiment of the present invention, the P2X₇ receptor antagonist is an adamantyl derivative of formula

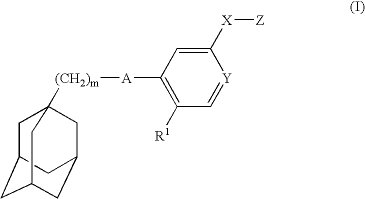

wherein m represents 1, 2 or 3;
A represents C(O)NH or NHC(O);
Y represents N or CH;
X represents a bond, CO, (CH₂)₁₋₆, O(CH₂)₁₋₆, (CH₂)₁₋₆NH(CH₂)₁₋₆, (CH₂)₁₋₆O(CH₂)₁₋₆ or NH(CH₂)₁₋₆;
Z represents NR²R³;
R¹ represents halogen, cyano, nitro, amino, hydroxyl, C₁-C₆ alkyl or C₃-C₈ cycloalkyl, which alkyl or cycloalkyl group can be optionally substituted by one or more fluorine atoms;

R² and R³ each independently represent a hydrogen atom, C₁-C₆ alkyl or C₃-C₈ cycloalkyl, which alkyl or cycloalkyl group can be optionally substituted by one or more groups selected from hydroxyl, halogen or C₁-C₆ alkoxy, or R² and R³ together with the nitrogen atom to which they are attached form a 3- to 9-membered saturated mono- or bicyclic heterocyclic ring comprising from 1 to 2 nitrogen atoms and optionally an oxygen atom, which heterocyclic ring can be optionally substituted by one or more groups selected from hydroxyl, halogen or C₁-C₆ alkoxy; or a pharmaceutically acceptable salt thereof.

In one aspect of this embodiment, in formula (I): m represents 1; A represents NHC(O); Y represents CH; X represents a bond, CO, CH₂, CH₂CH₂, CH₂CH₂CH₂, OCH₂, OCH₂CH₂ or OCH₂CH₂CH₂; Z represents NR²R³; R¹ represents chloro, bromo, fluoro, methyl or trifluoromethyl; and R² and R³ each independently represent a hydrogen atom or a C₁-C₄ alkyl group (e.g. methyl, ethyl, n-propyl or n-butyl) optionally substituted by hydroxyl or, alternatively, R² and R³ together with the nitrogen atom to which they are attached form a 9-oxa-3,7-diazabicyclo[3.3.1]non-3-yl group.

Adamantyl derivatives of formula (I) may be prepared according to known chemistry, for example by methods according or analogous to those described in WO 00/61569, WO 01/42194, WO 01/44170 and WO 03/41707.

The P2X₇ receptor antagonist used in the present invention may be capable of existing in all stereoisomeric forms including all geometric and optical isomers of the active ingredient and mixtures thereof including racemates. It will also be understood that certain P2X₇ receptor antagonists may exist in solvated, for example hydrated, as well as unsolvated forms, and the present invention encompasses all such solvated forms.

Pharmaceutically acceptable salts of P2X₇ antagonists of formula (I) include, where applicable, acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, hydroxynaphthalene-carboxylate or oleate salt; and salts prepared from pharmaceutically acceptable inorganic and organic bases. Salts derived from inorganic bases include aluminium, ammonium, calcium, copper, ferric, ferrous, lithium, magnesium, manganic, manganous, potassium, sodium, zinc and bismuth salts. Salts derived from pharmaceutically acceptable organic bases include salts of primary, secondary and tertiary amines, cyclic amines like arginine, betaine, choline and the like.

In another embodiment of the present invention, the P2X₇ receptor antagonist is selected from
2-Chloro-5-[[2-(2-hydroxy-ethylamino)-ethylamino]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,
   or a pharmaceutically acceptable salt thereof.

Examples of particular P2X₇ receptor antagonists that may be employed in accordance with the present invention include:—
2-Chloro-5-[[2-(2-hydroxy-ethylamino)-ethylamino]-methyl]-N-(tricyclo[3.3.1.1^{3,7}]dec-1-ylmethyl)-benzamide, dihydrochloride
2-Chloro-5-[3-[(3-hydroxypropyl)amino]propyl]-N-(tricyclo[3.3.1.1]dec-1-ylmethyl)-benzamide, hydrochloride
(R)-2-Chloro-5-[3-[(2-hydroxy-1-methylethyl)amino]propyl]-N-(tricyclo[3.3.1.1^{3,7}]dec-1-ylmethyl)-benzamide, hydrochloride
2-Chloro-5-[[2-[(2-hydroxyethyl)amino]ethoxy]methyl]-N-(tricyclo[3.3.1.1^{3,7}]dec-1-ylmethyl)-benzamide, acetate (1:1) salt
2-Chloro-5-[3-[3-(methylamino)propoxy]propyl]-N-(tricyclo[3.3.1.1^{3,7}]dec-1-ylmethyl)benzamide, hydrochloride
2-Chloro-5-[3-(3-hydroxy-propylamino)-propoxy]-N-(tricyclo[3.3.1.1^{3,7}]dec-1-ylmethyl)-benzamide, hydrochloride
2-Chloro-5-[2-(3-hydroxypropylamino)ethylamino]-N-(tricyclo[3.3.1.1^{3,7}]dec-1-ylmethyl)-benzamide, acetate (1:1) salt
2-Chloro-5-[2-(3-hydroxypropylsulfonyl)ethoxy]-N-(tricyclo[3.3.1.1^{3,7}]dec-1-ylmethyl)-benzamide
2-Chloro-5-[2-[2-[(2-hydroxyethyl)amino]ethoxy]ethoxy]-N-(tricyclo[3.3.1.1^{3,7}]dec-1-ylmethyl)-benzamide, hydrochloride
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide
2-Chloro-5-piperazin-1-ylmethyl-N-(tricyclo[3.3.1.1]dec-1-ylmethyl)-benzamide, dihydrochloride
2-Chloro-5-(4-piperidinyloxy)-N-(tricyclo[3.3.1.1^{3,7}]dec-1-ylmethyl)-benzamide, hydrochloride
2-Chloro-5-(2,5-diazabicyclo[2.2.1]hept-2-ylmethyl)-N-(tricyclo[3.3.1.1]dec-1-ylmethyl)-benzamide, hydrochloride
2-Chloro-5-(piperidin-4-ylsulfinyl)-N-(tricyclo[3.3.1.1^{3,7}]dec-1-ylmethyl)-benzamide
5-Chloro-2-[3-[(3-hydroxypropyl)amino]propyl]-N-(tricyclo[3.3.1.1^{3,7}]dec-1-ylmethyl)-4-pyridinecarboxamide,
5-Chloro-2-[3-(ethylamino)propyl]-N-(tricyclo[3.3.1.1^{3,7}]dec-1-ylmethyl)-4-pyridinecarboxamide, hydrochloride
5-Chloro-2-[3-[(2-hydroxyethyl)amino]propyl]-N-(tricyclo[3.3.1.1^{3,7}]dec-1-ylmethyl)-4-pyridinecarboxamide, hydrochloride
5-Chloro-2-[3-[[(2S)-2-hydroxypropyl]amino]propyl]-N-(tricyclo[3.3.1.1^{3,7}]dec-1-ylmethyl)-4-pyridinecarboxamide, dihydrochloride, and
N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride.

The second active ingredient in the combination of the present invention is a corticosetroid. The corticosteroid of the present invention may be any synthetic or naturally occurring corticosteroid. Examples of corticosteroids that may be used in accordance with the present invention include budesonide, fluticas one, mometas one, beclomethasone, ciclesonide, triamcinolone, flunisolide, zoticasone, flumoxonide, rofleponide, butixocort, prednisolone, prednisone, tipredane, steroid esters according to WO 2002/12265, WO 2002/12266 and WO 2002/88167 (I) e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3 S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, and steroid esters according to DE 4129535 (II).

In the context of the present specification, unless otherwise indicated any reference to a corticosteroid includes all active salts, solvates or derivatives that may be formed from said corticosteroid. Examples of possible salts or derivatives of corticosteroids include; sodium salts, sulphobenzoates, phosphates, isonicotinates, acetates, propionates, dihydrogen phosphates, palmitates, pivalates, furnarates and pharmaceutically acceptable esters (e.g. C₁-C₆ alkyl esters). Corticosteroids and active salts thereof may also be in the form of their solvates, e.g. hydrates.

In one embodiment of the present invention the corticosteroid is selected from budesonide, fluticasone (e.g. as propionate ester), mometasone (e.g. as furoate ester), beclomethasone (e.g. as 17-propionate or 17,21-dipropionate esters), ciclesonide, triamcinolone (e.g. as acetonide), flunisolide, rofleponide and butixocort (e.g. as propionate ester).

In one embodiment of the present invention the corticosteroid is budesonide. The chemical name for budesonide is 16,17-[butylidenebis(oxy)]-11,21-dihydroxy-pregna-1,4-diene-3,20-dione). Budesonide and its preparation is described, for example, in Arzneimittel-Forschung (1979), 29 (11), 1687-1690, DE 2,323,215 and U.S. Pat. No. 3,929,768. Presently available formulations of budesonide are marketed under the tradename ‘Entocort’.

The pharmaceutical product of the present invention may further optionally comprise, as a third active ingredient, a β₂-agonist. β₂-agonists (also known as beta2 (β₂) adrenoceptor agonists) may be used to alleviate symptoms of respiratory diseases by relaxing the bronchial smooth muscles, reducing airway obstruction, reducing lung hyperinflation and decreasing shortness of breath.

The β2-agonist of the present invention may be any compound or substance capable of stimulating the β2-receptor and acting as a bronchodilator. Examples of β2-agonists that may be used in the present invention include bambuterol, bitolterol, carbuterol, clenbuterol, fenoterol, formoterol, hexoprenaline, ibuterol, pirbuterol, procaterol, reproterol, salmeterol, sulphonterol, terbutaline, indacaterol and tolubuterol. In one aspect, the β₂-agonist of the invention is a long acting β₂-agonist, i.e. a β₂-agonist with activity that persists for more than 12 hours. Examples of long acting β₂-agonists include formoterol, bambuterol, salmeterol and indacaterol.

In the context of the present specification, unless otherwise stated, any reference to a β₂-agonist includes active salts, solvates or derivatives that may be formed from said β₂-agonist and any enantiomers and mixtures thereof. Examples of possible salts or derivatives of β₂-agonists are acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, and pharmaceutically acceptable esters (e.g. C₁-C₆ alkyl esters). The β₂-agonists may also be in the form of solvates, e.g. hydrates.

In an embodiment of the present invention, the O₂-agonist is formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. In one aspect of this embodiment, the β₂-agonist is formoterol fumarate.

It will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide and a mixture of such enantiomers, including a racemate.

In an embodiment of the present invention, there is provided a pharmaceutical product comprising, in combination, a first active ingredient which is a P2X₇ receptor antagonist, a second active ingredient which is budesonide and a third active ingredient which is formoterol.

The combination of the present invention may provide a beneficial therapeutic effect in the treatment of respiratory diseases. Examples of such possible effects include improvements in one or more of the following parameters: reducing inflammatory cell influx into the lung, mild and severe exacerbations, FEV, (forced expiratory volume in one second), vital capacity (VC), peak expiratory flow (PEF), symptom scores and Quality of Life.

The P2X₇ receptor antagonist (first active ingredient), corticosteroid (second active ingredient) and optionally β₂-agonist (third active ingredient) of the present invention may be administered simultaneously, sequentially or separately to treat respiratory diseases. By sequential it is meant that the active ingredients are administered, in any order, one immediately after the other. They may still have the desired effect if they are administered separately, but when administered in this manner they will generally be administered less than 4 hours apart, more conveniently less than two hours apart, more conveniently less than 30 minutes apart and most conveniently less than 10 minutes apart.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (to the lung and/or airways) in the form of solutions, suspensions, aerosols and dry powder formulations. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

In one embodiment of the present invention the active ingredients are administered via separate pharmaceutical preparations. This embodiment may be employed, for example, when the P2X₇ receptor antagonist is conveniently administered by oral administration, and the corticosteroid is conveniently administered by inhalation. In this embodiment the different pharmaceutical preparations of active ingredients may be administered simultaneously, sequentially or separately.

Therefore, in one aspect, the present invention provides a kit comprising a preparation of a first active ingredient which is a P2X₇ receptor antagonist, and a preparation of a second active ingredient which is a corticosteroid, and optionally instructions for the simultaneous, sequential or separate administration of the preparations to a patient in need thereof. The kit may further optionally comprise a preparation of a third active ingredient, which is a β₂-agonist.

In another embodiment the active ingredients may be administered via a single pharmaceutical composition. Therefore, the present invention further provides a pharmaceutical composition comprising, in admixture, a first active ingredient, which is a P2X₇ receptor antagonist, and a second active ingredient, which is a corticosteroid. The pharmaceutical composition may further optionally comprise a third active ingredient, which is a β₂-agonist.

The pharmaceutical compositions of the present invention may be prepared by mixing the P2X₇ receptor antagonist (first active ingredient) with a corticosteroid (second active ingredient), with a pharmaceutically acceptable adjuvant, diluent or carrier, and, optionally, a β₂-agonist (third active ingredient). Therefore, in a further aspect of the present invention there is provided a process for the preparation of a pharmaceutical composition, which comprises mixing a P2X₇ receptor antagonist with a corticosteroid, a pharmaceutically acceptable adjuvant, diluent or carrier, and, optionally, a β₂-agonist.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the P2X₇ receptor antagonist is administered via inhalation. When administered via inhalation the dose of the P2X₇ receptor antagonist will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In another embodiment of the present invention, the P2X₇ receptor antagonist is administered orally. Oral administration of the P2X₇ receptor antagonist may for example be used in a pharmaceutical product or kit wherein the other active ingredient(s) are administered by inhalation. When administered orally, satisfactory results will generally be obtained when the dose of the P2X₇ receptor antagonist is in the range of from 5 to 1000 milligram (mg), 5 to 800 mg, 5 to 600 mg, 5 to 300 mg, 5 to 400 mg, 5 to 300 mg, 5 to 200 mg, 5 to 100 mg, 5 to 50 mg, 20 to 1000 mg, 20 to 800 mg, 20 to 600 mg, 20 to 500 mg, 20 to 400 mg, 20 to 300 mg, 20 to 200 mg, 20 to 100 mg, 20 to 50 mg, 50 to 1000 mg, 50 to 800 mg, 50 to 600 mg, 50 to 500 mg, 50 to 400 mg, 50 to 300 mg, 50 to 200 mg, 50 to 100 mg, 100 to 1000 mg, 100 to 800 mg, 100 to 600 mg, 100 to 500 mg, 100 to 400 mg, 100 to 300 mg, or 100 to 200 mg. The dose will generally be administered from 1 to 4 times a day, conveniently once or twice a day, and most conveniently once a day.

In one embodiment of the present invention the corticosteroid is administered via inhalation. When administered via inhalation the dose of the corticosteroid will generally be in the range of from 0.1 μg to 5000 μg, 0.1 to 1000 μg, 0.1 to 500 μg, 0.1 to 100 μg) 0.1 to 50 μg, 5 μg to 5000 μg, 5 to 1000 μg, 5 to 500 μg, 5 to 100 μg, 5 to 50 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

When present, the β₂-agonist may conveniently be administered by inhalation. When administered via inhalation the dose of the β₂-agonist will generally be in the range of from 0.1 to 50 μg, 0.1 to 40 μg, 0.1 to 30 g, 0.1 to 20 μg, 0.1 to 10 μg, 5 to 10 μg, 5 to 50 μg, 5 to 40 μg, 5 to 30 μg, 5 to 20 μg, 5 to 10 g, 10 to 50 μg, 10 to 40 μg, 10 to 30 μg, or 10 to 20 μg. The dose will generally be administered from 1 to 4 times a day, conveniently once or twice a day, and most conveniently once a day.

In one embodiment, the present invention provides a pharmaceutical product comprising, in combination, a first active ingredient which is a P2X₇ receptor antagonist, and a second active ingredient which is a corticosteroid, and optionally a third active ingredient which is a β₂-agonist, wherein each active ingredient is formulated for inhaled administration.

In a further embodiment, the present invention provides a pharmaceutical product comprising, in combination, a first active ingredient which is a P2X₇ receptor antagonist, and a second active ingredient which is a corticosteroid, and optionally a third active ingredient which is a O₂-agonist, wherein the first active ingredient is formulated for oral administration, the second active ingredient is formulated for inhaled administration, and when present the third active ingredient is formulated for inhaled administration. In this embodiment the P2X₇ receptor antagonist (first active ingredient), corticosteroid (second active ingredient) and optionally β₂-agonist (third active ingredient) may be administered simultaneously, sequentially or separately to treat respiratory diseases. Conveniently the second and third active ingredients may be administered simultaneously in the form of a single pharmaceutical composition.

The active ingredients are conveniently administered via inhalation (e.g. topically to the lung and/or airways) in the form of solutions, suspensions, aerosols and dry powder formulations. For example metered dose inhaler devices may be used to administer the active ingredients, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The present invention further provides a pharmaceutical product, kit or pharmaceutical composition according to the invention for simultaneous, sequential or separate use in therapy.

The present invention further provides the use of a pharmaceutical product, kit or pharmaceutical composition according to the invention in the manufacture of a medicament for the treatment of a respiratory disease, in particular chronic obstructive pulmonary disease or asthma.

The present invention still further provides a method of treating a respiratory disease which comprises simultaneously, sequentially or separately administering:

(a) a (therapeutically effective) dose of a first active ingredient which is a P2X₇ receptor antagonist; and
(b) a (therapeutically effective) dose of a second active ingredient which is a corticosteroid; and optionally
(c) a (therapeutically effective) dose of a third active ingredient which is a β₂-agonist.; to a patient in need thereof.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

### Assay

The following P2X₇ antagonist may be employed in the Assay:—

P2X₇ antagonist 1.

### 1. N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride

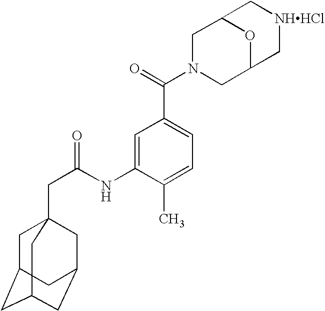

### a) 3-(4-Methyl-3-nitrobenzoyl)-7-(phenylmethyl)-9-oxa-3,7-diazabicyclo[3.3.1]nonane

Oxalyl chloride (9.6 ml) in dichloromethane (30 ml) was added dropwise over 45 minutes to an ice-cooled solution of 4-methyl-3-nitro-benzoic acid (10.0 g) in dichloromethane (320 ml) containing DMF (0.1 ml). The reaction mixture was stirred at room temperature for 1 hour then concentrated in vacuo. The acid chloride was taken into THF (320 ml) and cooled in an ice-bath before adding N,N-diisopropylethylamine (38 ml) then 3-(phenylmethyl)-9-oxa-3,7-diazabicyclo[3.3.1]nonane, dihydrochloride (16.0 g) (prepared as described in WO 01/028992) portionwise. The reaction was stirred for 18 hours then diluted with ethyl acetate (600 ml) and washed with water (2×200 ml) and saturated sodium bicarbonate (aq) (3×150 ml) then dried (MgSO₄), filtered and concentrated to afford the sub-titled compound (18.5 g).

m/z=382

### b) 3-(3-Amino-4-methylbenzoyl)-7-(phenylmethyl)-9-oxa-3,7-diazabicyclo[3.3.1]nonane

Reduced iron powder (7.9 g) was added over 15 minutes to a stirred solution of the product of step a) (18.0 g) and ammonium chloride (7.5 g) in ethanol/water (3:1, 320 ml) at 70° C. The reaction mixture was heated at reflux for 2 hours then filtered and concentrated in vacuo. The residue was taken into ethyl acetate (400 ml), washed with water (2×150 ml) then the organic phase dried (MgSO₄) and concentrated in vacuo to afford the sub-title compound (14.5 g).

m/z=352

### c) N-[2-Methyl-5-[[7-(phenylmethyl)-9-oxa-3,7-diazabicyclo[3.3.1]non-3-yl]carbonyl]phenyl]-tricyclo[3.3.1.1^{3,7}]decane-1-acetamide

Prepared by the method of step a) using 1-adamantaneacetic acid and the product of step b). Recrystallisation (ethyl acetate) afforded the sub-title compound.

m/z 528

### d) N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride

4M HCl in 1,4-dioxane (8 ml) was added to a solution of the product of step c) (13.0 g) in ethyl acetate (300 ml). The resulting precipitate was isolated by filtration then suspended in ethanol (300 ml) and 5% palladium on carbon (1.2 g) added. The reaction mixture was stirred under 3 atmospheres pressure of hydrogen for 36 hours. Methanol was then added under an atmosphere of nitrogen, then the catalyst removed by filtration and the filtrate concentrated in vacuo. Recrystallisation (isopropanol:methanol 25:1, 800 ml) gave the title compound (9.1 g).

m/z 438 (M+H)⁺

δ_{H} (400 MHz, d₆-DMSO, Me₄Si, 90° C.) 9.06 (1H, s), 7.64 (1H, s), 7.25 (1H, m), 7.19 (1H, m), 4.15 (2H, s), 3.96 (2H, d, J 14 Hz), 3.35-3.23 (6H, m), 2.26 (3H, s), 2.14 (2H, s), 1.96 (3H, br s), 1.69-1.62 (12H, m).

### Inflammatory Cell Influx Assay

The production of inflammatory cytokine interleukin (IL)-1 is increased in lungs of patients with COPD or asthma, and induction of interleukin-1β (IL-1β) in the lungs of adult mice has been shown to cause pulmonary inflammation, distal airspace enlargement consistent with emphysema, and other responses characteristic of respiratory disease (see Lappalainen et al, J. Respir. Cell Mol. Biol. 2005 April; 32(4), 311-8)

The effect of P2X₇ receptor antagonist 1 (N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride) and budesonide, and their combination, on cytokine levels may be assayed by measuring the effect on the levels of interleukin-1β (IL-1β) in broncholalveolar lavage (BAL) fluid of rats sensitised with ovalbumnin (subcutaneous) and challenged with ovalbumin (aerosol) (OVA). [N=8 rats per treatment group].

### Methodology

### Study Schedule

The assay may be carried out according to the study schedule set out in Table 1.

TABLE 1
Study schedule.

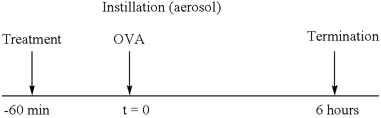

Treatments: At time point −60 minutes, rats were intratracheally instilled with suspensions containing i) a concentration of budesonide, or ii) a concentration of 1, or iii) a concentration of budesonide and a concentration of 1, each in a suitable vehicle. The control was ovalbumin challenge with vehicle treatment.

OVA aerosol challenge: A concentration of OVA (made up in saline) was given as a whole body aerosol challenge for 15 minutes to rats in sealed Perspex boxes at time point zero.

Bronchoalveolar ravage (BAL): 6 hours following OVA challenge rats were terminally anaesthetized and BAL was performed three times with 3.3 mls of phosphate buffer saline. The BAL fluid was centrifuged, and the supernatant removed and stored at −80° C. IL-1 levels were then measured using a commercially available rat IL-1β ELISA kit from R&D systems.

1. A pharmaceutical product comprising, in combination, a first active ingredient which is a P2X₇ receptor antagonist, and a second active ingredient which is a corticosteroid.

2. A product according to claim 1 wherein the P2X₇ receptor antagonist is a compound of formula 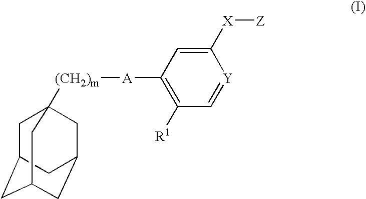
wherein m represents 1, 2 or 3;
A represents C(O)NH or NHC(O);
Y represents N or CH;
X represents a bond, CO, (CH₂)₁₋₆, O(CH₂)₁₋₆, (CH₂)₁₋₆NH(CH₂)₁₋₆, (CH₂)₁₋₆, O(CH₂)₁₋₆ or NH(CH₂)₁₋₆;
Z represents NR²R³;
R¹ represents halogen, cyano, nitro, amino, hydroxyl, C₁-C₆ alkyl or C₃-C₈ cycloalkyl, which alkyl or cycloalkyl group can be optionally substituted by one or more fluorine atoms;
R² and R³ each independently represent a hydrogen atom, C₁-C₆ alkyl or C₃-C₈ cycloalkyl, which alkyl or cycloalkyl group can be optionally substituted by one or more groups selected from hydroxyl, halogen or C₁-C₆ alkoxy,
or R² and R³ together with the nitrogen atom to which they are attached form a 3- to 9-membered saturated mono- or bicyclic heterocyclic ring comprising from 1 to 2 nitrogen atoms and optionally an oxygen atom, which heterocyclic ring can be optionally substituted by one or more groups selected from hydroxyl, halogen or C₁-C₆ alkoxy;
or a pharmaceutically acceptable salt thereof.

3. A product according to claim 1 wherein the P2X₇ receptor antagonist is selected from:
2-Chloro-5-[[2-(2-hydroxy-ethylamino)-ethylamino]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,
or a pharmaceutically acceptable salt of any one thereof.

4. A product according to claim 1, wherein the corticosteroid is budesonide.

5. A product according to claim 1, which further comprises a third active ingredient which is a β₂-agonist.

6. A product according to claim 5, in which the third active ingredient is formoterol.

7. (canceled)

8. The method according to claim 10, wherein the respiratory disease is chronic obstructive pulmonary disease.

9. The method according to claim 10, wherein the respiratory disease is asthma.

10. A method of treating a respiratory disease, which method comprises simultaneously, sequentially or separately administering:
(a) a (therapeutically effective) dose of a first active ingredient which is a P2X₇ receptor antagonist;
(b) a (therapeutically effective) dose of a second active ingredient which is a corticosteroid; and optionally
(c) a (therapeutically effective) dose of a third active ingredient which is a β2-agonist; to a patient in need thereof.

11. A kit comprising a preparation of a first active ingredient which is a P2X₇ receptor antagonist and a preparation of a second active ingredient which is a corticosteroid and optionally instructions for the simultaneous, sequential or separate administration of the preparations to a patient in need thereof.

12. A kit according to claim 11 wherein the P2X₇ receptor antagonist is selected from:
2-Chloro-5-[[2-(2-hydroxy-ethylamino)-ethylamino]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,
or a pharmaceutically acceptable salt of any one thereof.

13. A kit according to claim 11, wherein the corticosteroid is budesonide.

14. A kit according to claim 11, which further comprises a preparation of a third active ingredient which is a β₂-agonist.

15. A kit according to claim 14, in which the third active ingredient is formoterol.

16. A pharmaceutical composition comprising, in admixture, a first active ingredient which is a P2X₇ receptor antagonist, and a second active ingredient which is a corticosteroid.

17. A composition according to claim 16 wherein the P2X₇ receptor antagonist is selected from:
2-Chloro-5-[[2-(2-hydroxy-ethylamino)-ethylamino]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,
or a pharmaceutically acceptable salt of any one thereof.

18. A composition according to claim 16, wherein the corticosteroid is budesonide.

19. A composition according to claim 16, which further comprises a third active ingredient which is a β₂-agonist.

20. A composition according to claim 19, in which the third active ingredient is formoterol.
